(19) United States
(12) Patent Application Publication
(10) Pub. No.: US 2012/0330085 A1
(43) Pub. Date: Dec. 27, 2012

(54) SYSTEM AND METHOD FOR CALIBRATING AND POSITIONING A RADIATION THERAPY TREATMENT TABLE

(76) Inventors: Kenneth J. Ruchala, Madison, WI (US); Adam Schloesser, Mount Horeb, WI (US); Eric Schloesser, Mount Horeb, WI (US); David Murray, Tauranga (NZ)

(21) Appl. No.: 13/545,545

(22) Filed: Jul. 10, 2012

Related U.S. Application Data
(63) Continuation of application No. 12/651,680, filed on Jan. 4, 2010, now abandoned.
Application No. 12/651,680 is a continuation of application No. 10/577,567, filed on Mar. 23, 2007, now abandoned.
(60) Provisional application No. 60/515,481, filed on Oct. 29, 2003.

(51) Int. Cl. A61N 5/10

(52) U.S. Cl. 600/1

(57) ABSTRACT

A system and method for calibrating and positioning a radiation therapy treatment table with a high degree of precision and accuracy. The treatment table includes radiographic indexing markers embedded or inlaid therein. The radiographic indexing markers can include a density different from density of the table material, or a radio-frequency or magnetic contrasting matter, something that the imaging system and detector of the radiation therapy system can detect, so that when scanned, with or without a patient or phantom on the table, the precise location of the patient treatment table in the x, y and z planes is known. This is important so that the table may be reconstructed, alone or with a phantom on it, by the radiation therapy system software, providing a very good indication of where the table top is prior to treatment.

### RELATED APPLICATIONS

This application is a continuation of and claims priority to U.S. patent application Ser. No. 12/651,680, filed on Jan. 4, 2010, which is a continuation of U.S. patent application Ser. No. 10/577,567, filed on Mar. 23, 2007, which is a national stage filing under 35 U.S.C. §371 of International Application No. PCT/US04/36416, filed on Oct. 29, 2004, and claims priority to U.S. Provisional Patent Application No. 60/515,481, filed on Oct. 29, 2003. The entire contents of each of these applications are incorporated herein by reference.

### BACKGROUND OF THE INVENTION

The present invention relates generally to radiographic indexing of a radiation therapy treatment table, and more particularly to a system and method for calibrating and positioning a radiation therapy treatment table with a high degree of precision and accuracy through the use of radiographic indexing markers embedded in the treatment table.

Radiation therapy treatment tables are conventionally and typically used to hold the patient in a substantially rigid position, while the patient is being exposed to a radiation source. Typically, the radiation source is mounted above the treatment table and movable around the table so that the patient can be exposed to radiation from all possible angles, and allowing for purposefully directed radiation at the precise tumor site needed to destroy the tumor and minimize the exposure to healthy tissue.

In radiation therapy treatment of patients, it is imperative that the patient be held in a correct position relative to the radiotherapy machine in order to attain the expected treatment result without damaging healthy tissue outside of the treatment area. A need exists for a patient positioning system that is used to accurately and reproducibly position a patient on a table for radiation therapy, diagnostic imaging, surgery, and other medical procedures.

### SUMMARY OF THE INVENTION

The present invention provides a system and method for calibrating and positioning a radiation therapy treatment table with a high degree of precision and accuracy. The present invention provides for embedding or inlaying material for radiographic referencing into the patient treatment table of a radiation therapy system. The material is preferably a radiological marker or indexing material that has a density different from density of the table material, or a radio-frequency or magnetic contrasting matter, something that the imaging system and detector of the radiation therapy system can detect, so that when scanned, with or without a patient or phantom on the table, the precise location of the patient treatment table in the x, y and z planes is known. The material may also have the ability to be visually seen by the technician, doctor, physicist or other health care provider conducting the procedure. This is important so that the table may be reconstructed, alone or with a phantom on it, by the radiation therapy system software, providing a very good indication of where the table top is prior to treatment.

The present invention provides for calibrating the location and position of the treatment table, and also for accurately positioning a patient on the treatment table. For example, the present invention is capable of providing methods for aligning the table with the bore, detecting the scan plane, determining the slice-sensitivity profile for computed tomography (CT) and other modality images, and aligning lasers with the virtual scan plane.

The present invention also provides methods to verify quality assurance (QA) and patient procedures. For example, the present invention is capable of providing methods for verifying the table speed and/or position and table-bore collision avoidance.

In addition to the calibration and QA uses described above, the present invention may also be used for: (a) improving or simplifying the setup of the patient on the table; (b) improving or simplifying the placement of patient fixation devices on the table; and (c) identifying coordinate transformations between patient space and fixation-device space. For example, the radiation therapy system could convert a set of patient adjustments detected in a CT image to proper translations or rotations on a given fixation device.

As mentioned above, the present invention comprises a patient treatment table having a radiological marker or indexing matrix embedded or inlaid into the patient treatment table of a radiation therapy system. The inlaid radiological marker or indexing matrix may be placed and spaced in any configuration in the table. The markers can be permanent or replaceable. The radiological markers or indexing matrix may be actual material or voids that provide contrasting densities in the treatment table that are detectable. The present invention includes various styles and forms of radiological markers and in various configurations.

The marker or indexing matrix is comprised of contrasting high or low density material in the form of solids (e.g. wire, pellets, balls, paste, etc.), liquids (e.g. column of water, bubble-level, etc.), disjoint solids (e.g. strings of marker beads, etc.) that are inlaid into or fixed onto the sides, top or bottom of the patient treatment table. These density markers can either be actual objects or the absence or objects (e.g. voids).

In one embodiment, the markers are protrusions from the table, allowing for patient fixation devices to be connected to the protrusions. In another embodiment, the markers are flush with the sides or surfaces of the table. In yet another embodiment, the markers are indentations or grooves in the table. In still another embodiment, the markers are embedded at a depth into the table.

The markers may be individual markers or may be arranged in sets of two or three, or more, and potentially utilize the sides and/or surfaces of the treatment table. The markers may also be permanently fixed or removable from the treatment table, to minimize interference with dose delivery. The markers may also be interchangeable with different sets or subsets utilized for different objectives.

In another embodiment, the markers are magnetic, or include magnets or sensors that are used for positioning patients and/or fixation devices on the treatment table.

In another embodiment, the markers emit radio frequency (RF) signals which are detected using RF sensitive detection devices. The markers of the present invention may also be RF detectors for detecting RF signals.

In yet another embodiment, the markers include lasers that can be used for any of the above purposes, including the positioning of patients and/or fixation devices on the table, avoidance of table collisions, system setup and calibration, etc. The markers, including lasers, may send and/or receive signals with other fixtures in the room to detect changes in table height or deformations in the table, such as sagging. This information can be used to automatically, semi-automatically, or manually adjust the table position before or during treatment procedures.

The markers may also be used for calibration and verification of table speed, table position and of the virtual isocenter. Treatment accuracy is maintained by aligning the linear accelerator's isocenter to a precise point within the patient. The isocenter is a single reference point in the x, y and z planes of the Cartesian coordinate system which serves to orient the radiation beams to the proper coordinates of the slices of the tumor in the patient.

In further embodiments, the present invention comprises radiographic plugs, contrast inserts or resolution inserts that are inserted into openings in the patient treatment table. In an alternative embodiment, the contrast inserts or resolution inserts could also be embedded or molded into the table. The plugs or inserts are preferably used for calibrating the imaging system or dose delivery, and positioning the patient on the table.

Various other features, objects, and advantages of the invention will be made apparent to those skilled in the art from the accompanying drawings and detailed description thereof.

### BRIEF DESCRIPTION OF THE DRAWINGS

FIG. 1 is a perspective, cut-away view of a radiation therapy system providing for the acquisition of radiographic projections and for the generation of high energy radiation therapy beams and showing a patient table for supporting a patient thereon;

FIG. 2 is a perspective view and detail of an embodiment of a radiographic marker indexing grid embedded or inlaid into the patient table top;

FIG. 3 is a top plan view and detail of another embodiment of a radiographic marker indexing grid embedded or inlaid into the patient table top; and

FIG. 4 is a cross-sectional view and detail of the patient table top of FIG. 3 taken along line 4-4.

### DETAILED DESCRIPTION

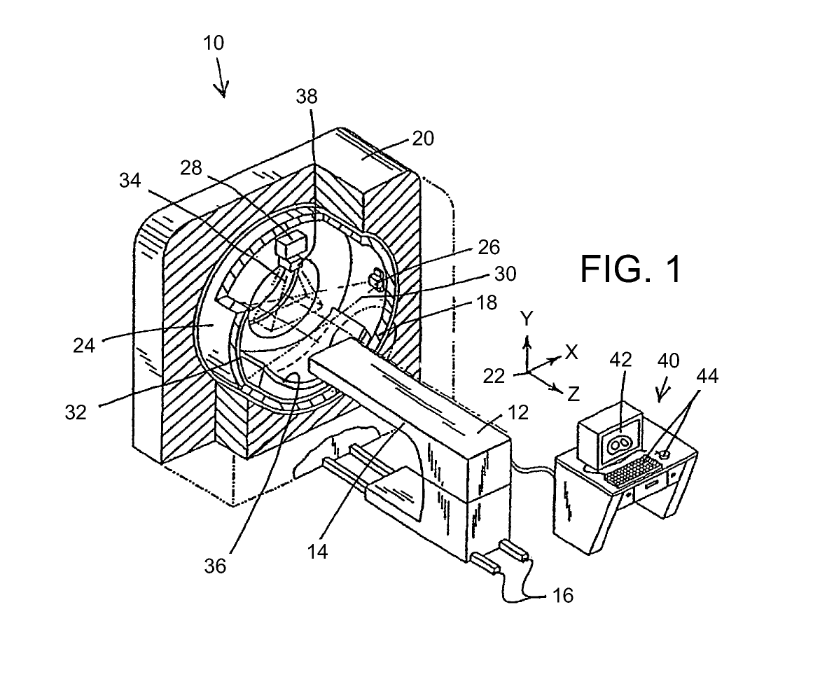

Referring now to the drawings, FIG. 1 illustrates a radiation therapy machine 10 suitable for use with the present invention. The radiation therapy machine 10 preferably includes a radiotranslucent table 12 having a cantilevered top 14. The table top 14 is received within a bore 18 of an annular housing 20 of the radiation therapy machine 10 with movement of the table 12 along tracks 16 extending along a longitudinal axis, such as the z-axis of a Cartesian coordinate system 22. The table 12 is preferably disposed along a translation axis and may slide along that axis through the bore 18 passing first the front surface and then the rear surface. The table 12 is preferably supported along the guide tracks 16 and moved by a motorized drive, such as is well known in the art, so that its position may be controlled by a computer as will be described. The table 12 also preferably includes an internal track assembly and elevator (not shown) to allow adjustment of the table top 14 in a lateral horizontal position (indicated by the x-axis of the coordinate system 22) and a vertically (indicated by the y-axis of the coordinate system 22). Motion in the lateral (x) and vertical (y) directions are limited by the diameter of the bore 18.

A rotating gantry 24, coaxial with the bore 18 and positioned within the housing 20, supports an x-ray source 26 and a high energy radiation source 28 on its inner surface. The x-ray source 26 and the radiation source 28 rotate with the gantry 24 about a center of rotation near the top of the patient table 12 when the table top 14 is positioned within the bore 18.

The x-ray source 26 is collimated to produce a fan beam 30 lying generally within the x-y plane and crossing the bore 18 and thus the table top 14 when the table top is positioned within the bore 18. The fan beam 30 diverges about a central axis whose angle is controlled by the position of the gantry 24. After exiting the table top 14, the fan beam 30 is received by a linear array detector 32 positioned diametrically across from the x-ray source 26. Thus, the rotating gantry 24 permits fan beam radiographic projections of a patient on the table top 14 to be acquired at a variety of angles about the patient during the treatment process.

The radiation source 28 is mounted so as to project a fan beam of high energy radiation 34, similar to the fan beam 30, but crossing fan beam 30 at right angles so as to be received on the other side of the gantry 24 by a radiation detector and stop 36. Therefore, the x-ray can be taken of a region prior to application of radiation. The radiation source 28 has a collimator 38 mounted in front of it to divide the fan beam of high energy radiation 34 into multiple adjacent rays whose intensity may be individually controlled to adjust the fluence of radiation. The location of the radiation source 28 and x-ray source 26 are precisely characterized so that images obtained from the x-ray source 26 may be used to aim the radiation source 28.

The radiation therapy machine 10 further includes a computer 40 having a display screen 42 and user entry input devices 44, well known in the art, that is connected to the radiation therapy machine 10 to control motion of the table 12 and to coordinate operation of the gantry 24 together with the radiation source 28 and x-ray source 26 and to collect data from the linear array detector 32 during a scan of a patient according to methods well known in the art.

The present invention provides radiographic indexing marker embedded or inlaid in the patient treatment table. The radiographic indexing markers are preferably used for positional calibration of the patient treatment table. The present invention comprises a patient treatment table having at least one radiological marker or indexing matrix embedded or inlaid into the patient treatment table of a radiation therapy system. The radiological marker or indexing matrix may be placed and spaced in any configuration in the table. The inlaid radiological marker or indexing matrix may be actual material or voids that provide contrasting densities in the treatment table that are detectable. The marker or indexing matrix is comprised of contrasting high or low density material in the form of solids (e.g. wire, pellets, balls, paste, etc.), liquids (e.g. column of water, bubble-level, etc.), disjoint-solids (e.g. strings of marker beads, etc.) that are inlaid into or fixed onto the sides, top or bottom of the patient treatment table. These density markers can either be actual objects or the absence or objects (e.g. voids). The present invention includes various styles and forms of radiological markers and in various configurations.

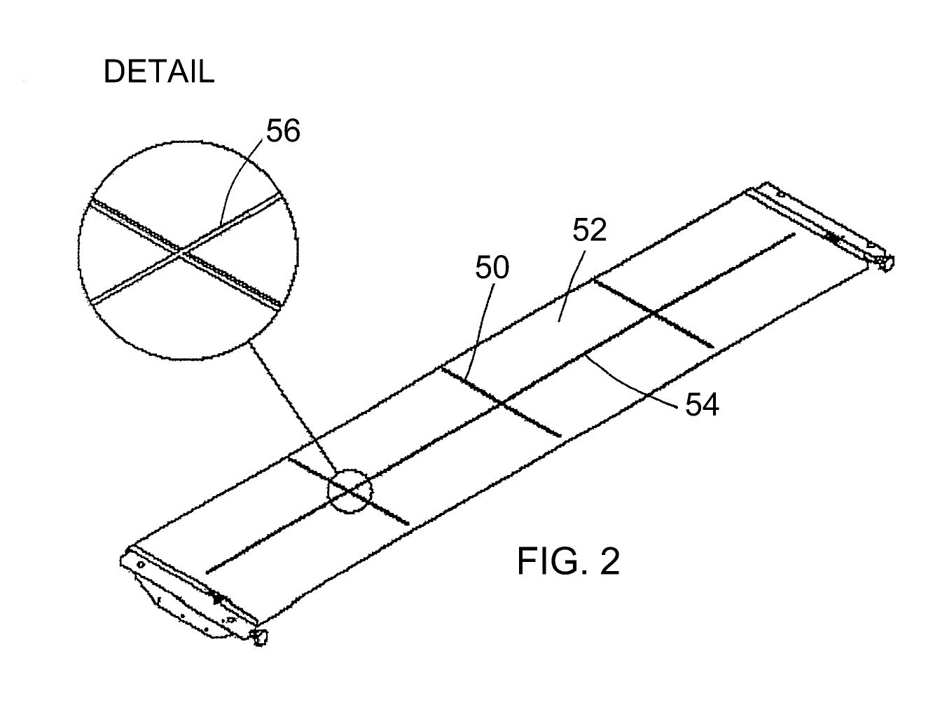

FIG. 2 is a perspective view and detail of an embodiment of a radiographic marker indexing grid 50 embedded or inlaid into the patient table top 52 of the radiation therapy system. The present invention preferably provides a high density material contrasted with the typical low density patient table top. In this embodiment, a wire inlay crosses latitudinally and longitudinally forming a matrix grid 54 across the patient table top 52. FIG. 2 shows a basic indexing grid, but the grid of the present invention is preferably significantly more complex to achieve accurate resolution in three dimensional space. The matrix grid 54 of radiological material inlaid or embedded in the table top 52 is preferably a three-dimensional material inlaid or embedded into the table top 52 that would allow for table indexing. For example, the detail shown in FIG. 2 shows a three-dimensional cross-hair 56 as part of the matrix grid of marker material inlaid or embedded in the table top 52. The wire inlays are shown as crossing at right angles. The wire inlays may also cross at other angles, such as forming a v-grid across the patient table. This allows for the imaging system of the radiation therapy system to detect the distances between markers and reference the patient table position. In a preferred embodiment, the marker material may be tungsten wire crisscrossed latitudinally and longitudinally across the patient table top 52. In addition, other more complex matrix grids or reference points may be inlaid or embedded into the table top 52 for more accuracy and precision of table top calibration and positioning.

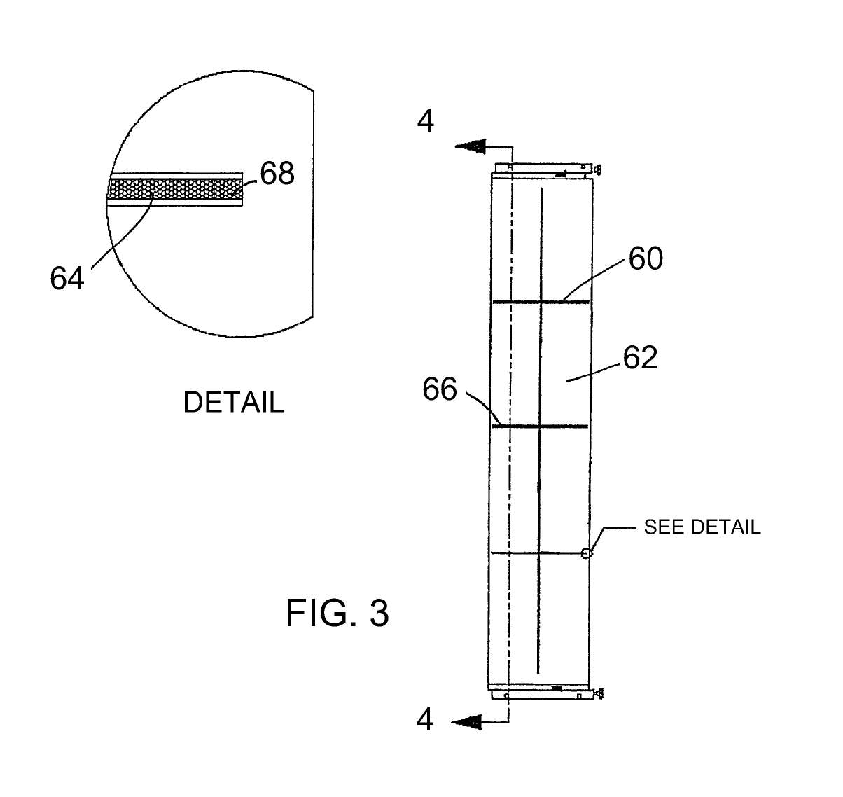

FIG. 3 is a top plan view and detail of another embodiment of a radiographic marker indexing grid 60 embedded or inlaid into the patient table top 62 of the radiation therapy system. In this embodiment, a plurality of contrasting density material 64 is embedded or inlaid in the patient table top 62 forming a matrix grid 66 across and/or through the patient table top 62. The matrix grid 66 of radiological material 64 inlaid or embedded in the table top 62 is preferably a three-dimensional material inlaid or embedded into the table top 62 that would allow for table indexing. For example, the detail shown in FIG. 3 shows a three-dimensional layer 68 of marker material inlaid or embedded in the table top 62. The three-dimensional layer 68 of marker material, may be a high density wire inlay, which when imaged, contrasts with the scanned body structure of a patient laying on the table top 62. In addition, other more complex matrix grids or reference points may be inlaid or embedded into the table top 62 for more accuracy and precision of table top calibration and positioning.

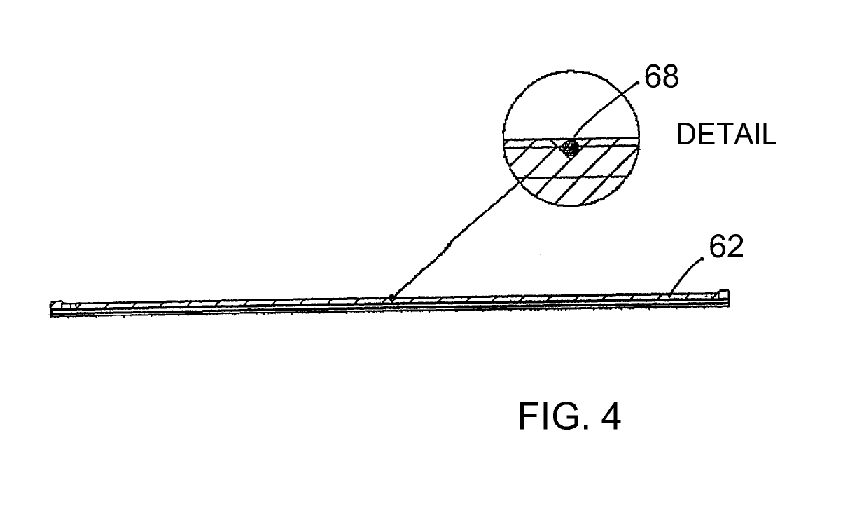

FIG. 4 is a cross-sectional view and detail of the patient table top 62 of FIG. 3 taken along line 4-4 showing a three-dimensional layer 68 of marker material inlaid or embedded in the table top 62 of the patient treatment table. For example, the three-dimensional layer of marker material may be an embedded tungsten radiographic marker.

In another embodiment, the markers are protrusions from the table, allowing for patient fixation devices to be connected to the protrusions. In another embodiment, the markers are flush with the sides or surfaces of the table. In yet another embodiment, the markers are indentations or grooves in the table. In still another embodiment, the markers are embedded at a depth into the table. The markers may be individual markers or may be arranged in sets of two or three, or more, and potentially utilize the sides and/or surfaces of the treatment table. The markers may also be permanently fixed or removable from the treatment table, to minimize interference with dose delivery. The markers may also be interchangeable with different sets or subsets utilized for different objectives.

In another embodiment, the markers are magnetic, or include magnets or sensors that are used for positioning patients and/or fixation devices on the treatment table. In still another embodiment, the markers emit radio frequency (RF) signals which are detected using RF sensitive detection devices. The markers of the present invention may also be RF detectors for detecting RF signals.

In yet another embodiment, the markers include lasers that can be used for any of the above purposes, including the positioning of patients and/or fixation devices on the table, avoidance of table collisions, system setup and calibration, etc. The markers, including lasers, may send and/or receive signals with other fixtures in the room to detect changes in table height or deformations in the table, such as sagging. This information can be used to automatically, semi-automatically, or manually adjust the table position before or during treatment procedures.

The markers can also include TLDs, MOSFETs, diodes, or other dosimetric devices used to measure dose during the treatment or scan procedures. Use of these devices can be integrated into QA, dose reconstruction, treatment planning, or other related applications.

The markers may also be used for calibration and verification of table speed, table position and of the virtual isocenter. Treatment accuracy is maintained by aligning the linear accelerator's isocenter to a precise point within the patient. The isocenter is a single reference point in the x-y-z planes which serves to orient the radiation beams to the proper coordinates of the slices of the tumor in the patient.

In addition to the embodiments shown, the present invention comprises radiographic plugs that are inserted into the table that are used for calibrating the imaging system. The plugs would preferably have a density different than bone, tissue or water. The radiographic plugs would preferably be inserted into a row of cylindrical holes at the ends of the table.

The plugs would generally be used to calibrate the imaging system. You would take an image of the table and make sure that you see the plugs where you expect to see them. The plugs are preferably used for density or imaging calibration. The plugs may be the same or a different density from the table material. The plugs are generally available from such manufacturers as Standard Imaging or Gammex.

In another embodiment, either contrast inserts or resolution inserts would be inserted into the openings described above. In an alternative embodiment, the contrast inserts or resolution inserts could also be embedded or molded into the table. The resolution inserts would preferably be a set of wires or openings based on some even or uneven spacing. These inserts would be used for calibrating the imaging system, and possibly positioning of the patient on the table.

In another embodiment, the present invention comprises a substantially flat member that includes radiographic indexing markers and is placed on and/or attaches to the patient treatment table for positional calibration of the treatment table.

The markers of the present invention may interact with markers in or on the patient, or characteristics of the patient, to determine the patient position relative to the couch. This includes, but is not limited to, magnetic, RF, laser, and/or light signals.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A system for calibrating and positioning a radiation therapy treatment table, the system comprising:
at least one radiographic marker included in the radiation therapy treatment table;
a radiation source configured to transmit a radiation beam through the radiation therapy treatment table, the radiation beam defining a pathway, and a portion of the at least one radiographic marker positioned within the pathway of the radiation beam; and
a detector positioned opposite the radiation source and configured to receive the radiation beam after it passes through the radiation therapy treatment table, the detector configured to identify a position of the radiation therapy treatment table based on a location of the radiographic marker.

2. The system of claim 1, wherein the at least one radiographic marker has a density different from the density of the material of the radiation therapy treatment table.

3. The system of claim 1, wherein the at least one radiographic marker includes a high density material.

4. The system of claim 1, wherein the at least one radiographic marker includes a low density material.

5. The system of claim 1, wherein the at least one radiographic marker is permanently fixed in the radiation therapy treatment table.

6. The system of claim 1, wherein the at least one radiographic marker is removable from the radiation therapy treatment table.

7. The system of claim 1, wherein the at least one radiographic marker is embedded or inlaid into the radiation therapy treatment table.

8. The system of claim 1, wherein the at least one radiographic marker is an indentation or groove in the radiation therapy treatment table.

9. The system of claim 1, wherein the at least one radiographic marker is magnetic and wherein sensors are used for positioning a patient and/or a fixation device.

10. The system of claim 1, wherein the at least one radiographic marker is a laser.

11. The system of claim 1, wherein the at least one radiographic marker is an RF emitter.

12. The system of claim 1, wherein the at least one radiographic marker is an RF detector.

13. The system of claim 1, wherein the at least one radiographic marker comprises a solid.

14. The system of claim 1, wherein the at least one radiographic marker comprises a disjoint solid.

15. The system of claim 1, wherein the at least one radiographic marker comprises a liquid.

16. The system of claim 1, wherein the at least one radiographic marker is interchangeable with different sets or subsets of markers utilized for different objectives.

17. The system of claim 1, wherein the detector is further configured to determine whether the radiation therapy treatment table sags based on the location of the radiographic marker.

18. The system of claim 1, wherein the detector is further configured to communicate with a computer to adjust the position of the radiation therapy treatment table.

19. The system of claim 1, wherein the detector is further configured to determine whether the radiation therapy treatment table is deformed based on the location of the radiographic marker.

20. The system of claim 1, wherein the detector is further configured to communicate with a computer to determine one of table speed, table position, and virtual isocenter based on the location of the radiographic marker.

21. A system for calibrating and positioning a radiation therapy treatment table, the system comprising:
at least one radiographic marker included in the radiation therapy treatment table;
a radiation source configured to transmit a radiation beam through the radiation therapy treatment table; and
a detector positioned opposite the radiation source and configured to receive the radiation beam after it passes through the radiation therapy treatment table; and
a computer system in communication with the detector, the computer system configured to receive data from the detector related to position of the at least one radiographic marker to determine a time-varying position of the treatment table.
